(19) United States
(12) Patent Application Publication
(10) Pub. No.: US 2019/0366230 A1
(43) Pub. Date: Dec. 5, 2019

(54) HIGH INTENSITY TARGETING (HIT) SUPERCRITICAL FLUID EXTRACTION SYSTEM AND RELATED METHODS

(71) Applicants: Tomas Casals, Cleveland, OH (US); Shane Kenney, Meredith, NH (US); Ethan Moon, Worcester, MA (US)

(72) Inventors: Tomas Casals, Cleveland, OH (US); Shane Kenney, Meredith, NH (US); Ethan Moon, Worcester, MA (US)

(21) Appl. No.: 16/426,823

(22) Filed: May 30, 2019

Related U.S. Application Data
(60) Provisional application No. 62/678,232, filed on May 30, 2018.

(51) Int. Cl. B01D 11/02; B01D 15/08

(52) U.S. Cl. CPC B01D 11/0203; B01D 11/0207; B01D 11/028; B01D 11/0292; B01D 15/08; B01D 11/0219

(57) ABSTRACT

The present invention is directed to supercritical fluid extraction systems that provide methods of extraction that further reduce total extraction time and improve yield efficiency as compared with existing methods. Such systems and methods utilize substantially increased pressures and flow rates that afford high intensity targeting of extractable material using supercritical carbon dioxide. Surprisingly, the present invention has identified and demonstrated dramatic improvements in the rate and efficiency of dissolution of the target compounds by increasing the pressure of supercritical fluid extraction systems to several times industry standards (e.g., 1000 bar versus 150-300 bar).

### RELATED APPLICATIONS

This application claims priority to U.S. Provisional Patent Application No. 62/678,232 filed on May 30, 2018; the entirety of which is incorporated herein by reference.

### BACKGROUND OF THE INVENTION

Supercritical Fluid Extraction (SFE) is a well-known process for separating one component from another using one or more supercritical fluids as an extracting solvent. This type of extraction has been used to collect a desired product from both solid matrices and liquids. Supercritical carbon dioxide (CO₂) is the most used supercritical fluid, sometimes modified by co-solvents such as ethanol or methanol.

The general process of extraction is considered a diffusion-based process, in which a solvent is required to diffuse into a matrix with the extractable material diffusing out of the matrix into the solvent. Given that diffusivities are much faster in supercritical fluids than in liquids, the supercritical fluid extraction can occur in a relatively much faster timescale than liquid extraction. In addition, due to the lack of surface tension and negligible viscosities as compared to liquids, the supercritical fluid extraction solvent can penetrate further into the matrix that is inaccessible to liquids.

Extraction conditions for supercritical carbon dioxide are typically described with respect to critical temperature and pressure; for example, such extraction conditions are formed above the critical temperature of 31° C. and critical pressure of 74 bar (although addition of modifiers may slightly alter these). The properties of a supercritical fluid extraction have been shown to be capable of alteration by varying the pressure and temperature, allowing selective extraction in certain combinations.

Existing systems for SFE extraction standardly contain 1) a pump for CO₂, 2) a means for heating pressurized liquid CO₂ creating supercritical CO₂, 3) a pressure vessel to contain the material ranging from simple tubing to more sophisticated purpose built vessels with pressure fittings, and 4) a collecting vessel. Such systems operate by pumping liquid carbon dioxide into a heating zone to establish supercritical conditions, which then passes into the extraction pressure vessel, where it diffuses into the solid matrix and dissolves the material to be extracted. The dissolved material is then collected in a vessel during depressurizing conditions.

Due to the requirement of high pressures for botanical extractions, pressures from 150 bar to pressures of up to 350 bar have been used during such extractions with supercritical carbon dioxide. Solubility at pressures beyond which, particularly with higher throughput units, have been difficult to achieve given the challenges of the compressibility of supercritical carbon dioxide. Moreover, at higher pressures in this range the extracted material accumulates an increasing amount of additional unwanted compounds which are also extracted from the botanical material (i.e., typically large, high melting-temperature lipids, phospholipids, and chlorophylls), and which increase dramatically with pressures over 350 bar, introducing significant impurity profiles to the extracted material. Such impurity profiles require more post-processing in order to achieve an end product with similar characteristics, and such post-processing commonly results in the loss of significant amounts of the target compound, and therefore lower efficiency yield ratios of botanical material to target compound.

Furthermore, it is well-known that maintaining flow rates in these standard high pressure methods is difficult. In particular, maintaining high flow rates at higher pressures commonly results in the uncontrolled loss of carbon dioxide supply along with the substantial risk of freezing that occurs in the lines and valves. For this reason, under high pressure conditions used for botanical extractions, the most productive yields are achieved under pressures of 350 bar and have been achieved using flow rates sufficiently slow enough to reduce or eliminate icing and to achieve diffusion and extraction; typically on the order of about 10 hours. Moreover, newer methods and devices that work to tightly control temperature, pressure, and flow rate have been able to only slightly reduce this extraction time in some cases, while still allowing for efficient yield ratios.

As such, there remains a need for supercritical fluid extraction systems and related methods of extraction that further reduce total extraction time and improve yield efficiency.

### SUMMARY OF THE INVENTION

Accordingly, the present invention is directed to supercritical fluid extraction systems that provide methods of extraction that further reduce total extraction time and improve yield efficiency as compared with existing methods. Such systems and methods utilize substantially increased pressures and flow rates that afford high intensity targeting of extractable material using supercritical carbon dioxide. Surprisingly, the present invention has identified and demonstrated dramatic improvements in the rate and efficiency of dissolution of the target compounds by increasing the pressure of supercritical fluid extraction systems to several times industry standards (e.g., 1000 bar versus 150-300 bar).

As such, one aspect of the invention provides a high intensity targeting (HIT) supercritical fluid extraction system comprising a carbon dioxide source container; a first heat exchanging device operationally associated with the carbon dioxide source designed to reduce the temperature of the carbon dioxide to maintain the carbon dioxide as a liquid; a high intensity optimization pump system capable of maintaining a steady (e.g., controlled) flow rate of the liquid carbon dioxide at pressures greater than or equal to about 300 bar (e.g., greater than or equal to about 350 bar, e.g., greater than or equal to about 680 bar) resulting in highly pressurized liquid carbon dioxide suitable for achieving high intensity extraction targeting; a second heat exchanger device operationally associated with the high intensity optimization pump system designed to heat the highly pressurized liquid carbon dioxide to above its critical point, such that supercritical carbon dioxide is formed; an extraction vessel designed to hold a pre-determined amount of extractable material, and which is positioned to receive the supercritical carbon dioxide from said second heat exchanger, wherein the extraction vessel may be sealed with the extractable material and supercritical carbon dioxide under highly pressurized conditions to produce a target compound laden supercritical carbon dioxide through high intensity extraction targeting; a flow regulator device suitable for releasing the pressurized target compound laden supercritical carbon dioxide from the extraction vessel in a controlled manner; and a collector vessel designed to coalesce the target compound from the target compound laden supercritical carbon dioxide through expansion cooling, collecting the target compound and producing carbon dioxide gas.

Another aspect of the invention provides a method of high intensity targeting (HIT) supercritical fluid extraction comprising the steps of: providing carbon dioxide from a source container; reducing the temperature of the carbon dioxide to maintain the carbon dioxide as a liquid; pumping the carbon dioxide liquid to result in highly pressurized liquid carbon dioxide suitable for achieving high intensity extraction targeting by increasing the flow of the carbon dioxide, and maintaining a steady (e.g., controlled) flow rate of the liquid carbon dioxide at pressures greater than or equal to about 300 bar (e.g., greater than or equal to about 350 bar, e.g., greater than or equal to about 680 bar); heating the highly pressurized liquid carbon dioxide to above its critical point, such that supercritical carbon dioxide is formed; sealing an extractable material with the supercritical carbon dioxide under pressurized conditions to produce a target compound laden supercritical carbon dioxide through high intensity extraction targeting, and which is positioned to receive the supercritical carbon dioxide from said second heat exchanger; releasing the pressurized target compound laden supercritical carbon dioxide from the extraction vessel in a controlled manner by flow regulation; and collecting the target compound and producing carbon dioxide gas by coalescing the target compound from the target compound laden supercritical carbon dioxide through expansion cooling, such that high intensity supercritical fluid extraction of a target compound is achieved.

### BRIEF DESCRIPTION OF THE FIGURES

Advantages of the present apparatus will be apparent from the following detailed description, which description should be considered in combination with the accompanying figures, which are not intended to limit the scope of the invention in any way.

FIG. 1 depicts a schematic view of a high intensity targeting (HIT) supercritical fluid extraction system of certain embodiments of the present invention wherein.

FIG. 2 is an exploded view of the schematic of FIG. 1 focusing on a high intensity optimization pump system of certain embodiments of the present invention depicting two pumps, which together are capable of increasing the flow of the liquid carbon dioxide, and maintaining a flow rate capable of reaching pressures resulting in highly pressurized liquid carbon dioxide suitable for achieving high intensity extraction targeting.

FIG. 3A is an exploded view of the schematic of FIG. 1 focusing on the coalescing/impingement devices of certain embodiments of the present invention, depicting 3 Angstrom zeolite molecular sieve media. FIG. 3B is a more detailed side perspective view of the exploded view of FIG. 3A.

FIG. 4 depicts a side perspective view of an extraction vessel depicting a pin-retained closure of the present invention.

FIG. 5 depicts a side perspective view of a mechanical ratio BPR of the present invention that is piloted by the downstream pressure and used to adjust the supply of air pressure to the actuator.

FIG. 6 depicts a wedge ring seal geometry employed in certain embodiments of the present invention for the extraction vessel closure, requiring less fabrication and more rapid actuation than existing closure seal designs.

### DETAILED DESCRIPTION OF THE INVENTION

Existing systems of supercritical fluid extraction utilize a supply of liquefied CO₂ to generate a supply of CO₂ in the supercritical or near-supercritical (aka trans-critical) phase condition as a solvent to dissolve and extract certain chemical compounds. After increasing the pressure of the supply liquid to pressures above the critical point of the solvent using liquid phase pumps, and subsequently increasing the temperature of the pressurized liquid to above the critical point using a process fluid heater, this solvent is brought to temperature and pressure in a pressure vessel containing botanical material, referred to as the extraction vessel, from which the “target compounds” are to be extracted.

In contrast, and improvement thereon, the present invention is directed to supercritical fluid extraction systems that allow for methods of extraction that further reduce total extraction time and improve yield efficiency as compared with existing methods. Such systems and methods utilize substantially increased pressures and flow rates that afford high intensity targeting of extractable material using supercritical carbon dioxide, e.g., cannabinoids and terpenes from cannabis. Surprisingly, the present invention has identified and demonstrated that dramatic increases in the pressure of supercritical fluid extraction systems to several times industry standards (e.g., 1000 bar versus 150-300 bar) results in substantial improvements in the rate and efficiency of dissolution of the target compounds, e.g., from cannabis .

Extractions of cannabis, for example, have not been pursued in the marijuana industry under such high pressure conditions given substantial risk of freezing that occurs in the lines and valves. Further, even at moderately increased pressures, on the high end of the standard pressures previously attempted for such extractions, the desirable volatile terpene compounds of cannabis extractions have been considered lost at ‘high’ pressure. However, in light of the instant discovery and present disclosure, the observation of this loss of terpenes in the extracted end product is more likely attributed to loss during collector vessel depressurization (as light hydrocarbons are very soluble in gaseous CO₂), or loss during the requisite post-processing. In fact, it has now been discovered, and disclosed herein that at 1000 bar (or greater), the forces of solvation are such that the rates of dissolution for certain target compounds, such as from cannabis, are several times those at lower pressures. In particular, the efficiency of dissolution using the systems and methods of the present invention (in terms of percentage mass of target compound extracted from the organic material) exceeds any other commonly used solvent in cannabis extraction. Without wishing to be bound by theory, it is believed that this improved rate of dissolution is due to higher bond dipole-dipole forces being created in the solvent cavity.

As such, in effect, increasing the pressures, for example, to 1000 bar, allows the use of CO₂, which is traditionally a nonpolar solvent, as a highly polar solvent. In fact, by modulating pressure in this range, the polarity (and conversely the solvating capacity for certain target compounds) can be “tuned” to selectively extract different compounds. Moreover, given the ability of the systems and methods of the present invention to reach higher pressures than previously attainable, the tunability of the solvent becomes capable of modulation with the capability to selectively target compounds through the use of these tunable solvent characteristics. For example, the present invention substantially improves the efficacy of selectively targeting compounds by polar attributes of supercritical carbon dioxide, which is dependent on the systems and methods of the present invention in that a sufficient range of attributes is only available with pressures exceeding 1000 bar.

The present invention, including supercritical fluid extraction systems and methods will be described with reference to the following definitions that, for convenience, are set forth below. Unless otherwise specified, the below terms used herein are defined as follows:

### 1. Definitions

As used herein, the term “a,” “an,” “the” and similar terms used in the context of the present invention (especially in the context of the claims) are to be construed to cover both the singular and plural unless otherwise indicated herein or clearly contradicted by the context.

The term “automated” is used herein to describe a process that is automated or semi-automated. Automated processes do not contain steps that require a human operator to perform any steps. Semi-automated processes contain one or more steps that require a human operator; however, differ from manual processes by containing at least one step that does not require a human operator.

The language “botanical material” is art-recognized, and is used herein to describe an extractable material obtained from a plant. In certain embodiments of the present invention, the botanical material is cannabis.

The term “cannabis” is art-recognized, and describes a genus of flowering plant in the family Cannabaceae, including, for example, cannabis sativa, cannabis indica and cannabis ruderalis.

The term “capacity” is used herein to describe the amount of extracted target compound that may be produced by a given amount of solvent at a steady flow rate and definable pressure range in a given period. Accordingly, the increased capacities, e.g., substantially increased capacities, of the systems described herein, given their capability to operate under high intensity conditions, afford a reduction of the time needed for extraction (i.e., allow for quicker rates of extraction), in stark contrast to existing/known systems that result in null flow rates due to freezing and clogging at the pressures less than those of the high intensity conditions described herein. In certain embodiments, target compounds that might be incapable of extraction under existing/known conditions, are extractable under high intensity targeting conditions described herein and attainable by the systems of the present invention.

The language “extractable material” is used herein to describe any composition from which an element, constituent, or substance is derivable through the process of extraction using supercritical fluid extraction.

The language “high intensity” as used in the language “high intensity targeting” or “HIT,” is used herein to describe the combination of conditions suitable to improve extraction targeting of a compound of interest, e.g., from improving capacity (e.g., substantially improving capacity) of an extraction system to actually allowing extraction of a material that might not otherwise be possible. The combination of conditions for high intensity targeting of compounds for extraction specifically relate to the ability to maintain a steady (e.g., controlled) flow rate of the liquid carbon dioxide at pressures greater than or equal to about 300 bar (e.g., 350 bar, e.g., 680 bar) resulting in highly pressurized liquid carbon dioxide suitable for achieving extraction conditions of high intensity to extract target compounds at a higher capacity than existing extraction systems.

The term “laden” as used herein with respect to solvent in the language “target compound laden, describes solvent, e.g., supercritical CO₂ solvent, carrying compounds/solute dissolved in the solvent, e.g., from extractable material. For example, a solvent that is target compound laden carries target compound dissolved in the solvent.

The term “operationally associated” is used herein to describe items that are associated, connected, or related in such a manner as to achieve a common intended purpose of operation of the items together. For example, a heat exchanging device may be operationally associated with the carbon dioxide source such that the two components are connected in such a way, e.g., tubing or piping, as to reduce the temperature of the carbon dioxide to maintain the carbon dioxide as a liquid.

The term “substantially” is used herein to describe the state of being to a great or significant extent. For example, as used in the term “substantially reduced,” the term “substantially” would indicate a significant extent of reduction (i.e., compared to systems that are not capable of pumping highly pressurized liquid carbon dioxide in a manner suitable for achieving high intensity extraction targeting). Such reduction, includes, for example, about 50% or greater reduction of the extraction period, e.g., about 60% or greater reduction of the extraction period, e.g., about 70% or greater reduction of the extraction period, e.g., about 80% or greater reduction of the extraction period, e.g., about 90% or greater reduction of the extraction period, e.g., about 95% or greater reduction of the extraction period.

### II. High Intensity Targeting (HIT) Supercritical Extraction Systems of the Invention

The present invention provides supercritical extraction systems suitable for high intensity targeting of compounds from extractable material, e.g., botanical material. Components of the system comprise a carbon dioxide source container, a first heat exchanging device, a high intensity optimization pump system, a second heat exchanger device, an extraction vessel, a flow regulator device, and a collector vessel. As such, one embodiment of the present invention provides a high intensity targeting (HIT) supercritical fluid extraction system comprising
a carbon dioxide source container (e.g., comprising carbon dioxide, e.g., a vessel containing liquid CO₂);
a first heat exchanging device operationally associated with the carbon dioxide source designed to reduce the temperature of the carbon dioxide to maintain the carbon dioxide as a liquid (e.g., to about −15° C.), e.g., designed to receive carbon dioxide through a path directly from the carbon dioxide source, e.g., and further from the recycling path;
a high intensity optimization pump system capable of maintaining a steady (e.g., controlled) flow rate of the liquid carbon dioxide at pressures greater than or equal to about 300 bar (e.g., greater than or equal to about 350 bar, e.g., greater than or equal to about 680 bar) resulting in highly pressurized liquid carbon dioxide suitable for achieving high intensity extraction targeting;
a second heat exchanger device operationally associated with the high intensity optimization pump system designed to heat the highly pressurized liquid carbon dioxide to above its critical point, such that supercritical carbon dioxide is formed;
an extraction vessel designed to hold a pre-determined amount of extractable material, and which is positioned to receive the supercritical carbon dioxide from said second heat exchanger, wherein the extraction vessel may be sealed with the extractable material and supercritical carbon dioxide under highly pressurized conditions to produce a target compound laden supercritical carbon dioxide through high intensity extraction targeting;
a flow regulator device suitable for releasing the pressurized target compound laden supercritical carbon dioxide from the extraction vessel in a controlled manner; and
a collector vessel designed to coalesce the target compound from the target compound laden supercritical carbon dioxide through expansion cooling, collecting the target compound and producing carbon dioxide gas. In certain embodiments of the invention, the systems of the present invention
   may be used for (1) extraction of any extractable material, e.g., producing both organic and inorganic target compounds, or (2) may be used where the high intensity conditions generated by the system may be useful, e.g., where the vessel is used for exposure and/or decontamination. Extractable target compounds may include, but are not limited to a vitamin, an essential oil, a terpene/terpenoid, caffeine (for caffeine production, or decaffeinated organic matter, e.g., coffee or tea), a wax, a lipid, a carotenoid, a flavonoid, a phenol, an herbal distillate/supplement/nutraceutical, a colorant (dye), an aroma (perfume), a nanoparticle, a specialty lubricant, a petroleum slurry, or omega 3 fatty acids. The high intensity conditions generated by the system may be useful in separations, preservations, decontaminations, or sterilizations, including, but not limited to metallurgical separation, radioactive waste decontamination, removing/reducing organochloride pesticide, removing/reducing dirt/other waste from clothing as an alternative to using hazardous fluids in dry cleaning (i.e., using supercritical CO₂ as the cleaning agent), removing/reducing an environmental pollutant (water, soil, etc.), removing/reducing an archeological artifact contaminant, separating a pharmaceutical binder, removing/reducing a microorganism (e.g., a virus such as HIV, or a bacterium such as E.coli) for medical purposes, sterilization for bone transplant, device sterilization, antibody production/preservation, food preservation (e.g., vegetable, fruit, or meat preservation).

In certain embodiments of the present invention, the extractable material is a botanical material, e.g., cannabis. As such, in certain embodiments, the target compound may be selected from one or more cannabinoids. In particular embodiments, the target compound may be selected from the group of cannabinoids consisting of THC (Tetrahydrocannabinol), THCA (Tetrahydrocannabinolic acid), CBD (Cannabidiol), CBDA (Cannabidiolic Acid), CBN (Cannabinol), CBG (Cannabigerol), CBC (Cannabichromene), CBL (Cannabicyclol), CBV (Cannabivarin), THCV (Tetrahydrocannabivarin), CBDV (Cannabidivarin), CBCV (Cannabichromevarin), CBGV (Cannabigerovarin), CBGM (Cannabigerol Monomethyl Ether), CBE (Cannabielsoin), CBT (Cannabicitran), and any combination thereof. In specific embodiments, the target compound are cannabis derived terpenes or terpenoids

Extraction of specific compounds, in certain embodiments of the present invention, may be considered to be fractionation using solubility or dissolution (as opposed to boiling point/vapor pressure for thermal fractionation). In particular embodiments, this process is useful in scenarios such as biosynthesis post-processing where the target compounds and other process compounds have differing polarities or solubility in supercritical CO₂ (SCO₂), but similar boiling points. Thus, separating them using the tunable solvating characteristics in SCO₂ would be faster/easier/more effective than thermal fractionation/distillation. Moreover, with respect to cannabis, supercritical fluid fractionation using HIT would be highly beneficial given that thermal distillation/fractionation carries a higher level of loss, especially because many of the target compounds have very similar boiling points.

Advantages of the high intensity targeting (HIT) supercritical fluid extraction systems of the present invention include, but are not limited to, a substantial increase in throughput, increased efficiency of extraction, reduced run times (which, in turn, means reduced cost for product, i.e., less run time means increased product amount per maintenance cost investment, and reduced run times also means greater labor efficiency and increased safety), improved targeting of specific compounds or compound groups, e.g., where capability of extraction of compounds not otherwise extractable is afforded, improved reliability at all flow rates and pressures, increased labor efficiency, and any combination thereof. In particular embodiments, depending on the target compound (and whether or not the target compound was extractable without the systems and methods of the present invention), such reduction, includes, for example, about 85% or greater reduction of the extraction period of tetrahydrocannabinol or cannabidiol, or about 99% or greater reduction of the extraction period of carotenoids.

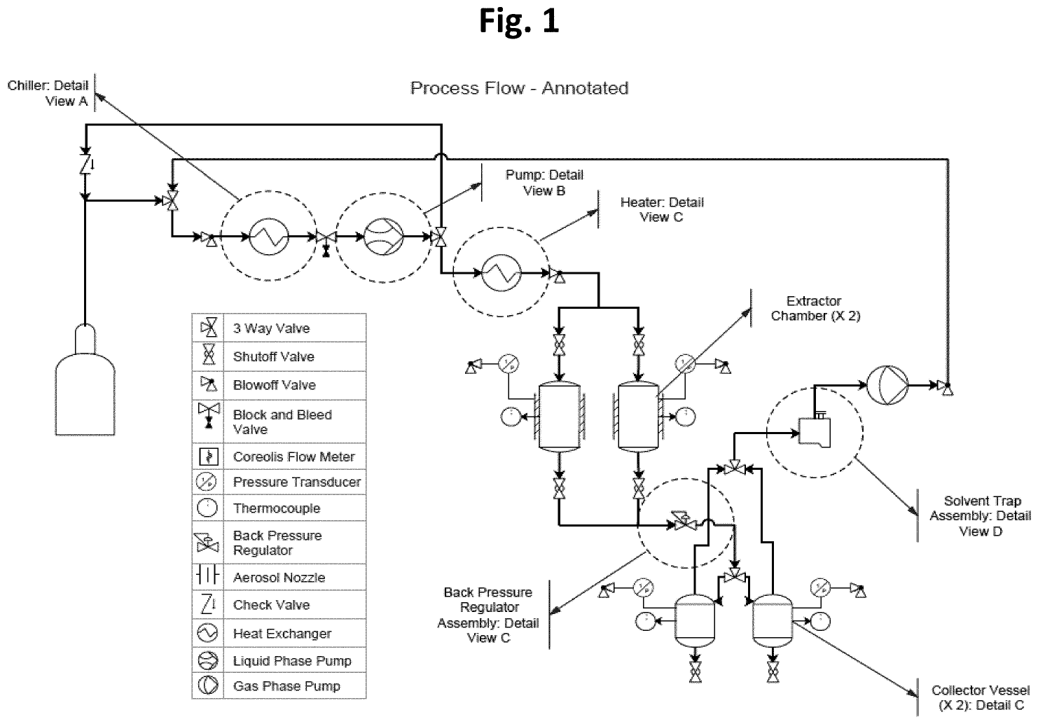

FIG. 1 depicts an exemplary embodiment of the HIT supercritical fluid extraction systems of the present invention. In one embodiment, liquefied CO₂ is stored in a carbon dioxide source container, depicted in FIG. 1 as a cylinder, e.g., at room temperature and under a pressure of about 62 bar (and therefore, in liquid form). The liquid may be supplied to the system through a dip tube in the cylinder. The carbon dioxide liquid flows through a first heat exchanger which is supplied with coolant to reduce the CO₂ temperature, e.g., to roughly −15 C. This cooling is performed to ensure that the CO₂ remains in a liquid state during pumping, especially at the higher cyclic rates of the pump (which allows faster system pressurization).

The cooled liquid CO₂ is pumped up to pressure using a high intensity optimization pump system, shown in FIG. 1 to include a parallel set of air driven piston-type liquid pumps. One pump has a lower compression ratio, corresponding to a higher flow rate, but which can only operate to a fraction of the ultimate pressure, i.e., this fraction is based on the compressibility of the CO₂, the desired ultimate pressure, pump ratios, and other variables such as air supply requirements. A second pump with a compression ratio capable of reaching ultimate pressure is used throughout the entire pumping process, and is responsible for the addition of CO₂ to the system at pressures above the stall point of the other pump. The pressurized liquid CO₂ flows through a second heat exchanger which is supplied heated transfer fluid. The heat transfer with the CO₂ increases the temperature to above the critical temperature where the CO₂ is said to be “supercritical”. This supercritical CO₂ flows into one or both extraction vessels shown in FIG. 1.

Once the ultimate, e.g., pre-determined, temperature and pressure are reached in the extraction vessel(s), the pumps and heater cease to operate and the supply side of the extraction vessel(s) is sealed. The supercritical CO₂ solvent and the extractable material located in the extraction vessel along with the supercritical CO₂ will be allowed to stand for a period of time, during which the target compounds will be dissolved into the supercritical CO₂ from the extractable material. After the set period of time, the vessel is depressurized, and CO₂ laden with target compounds flows through a flow regulator device, e.g., a micro-metering valve, into the collector vessel. The rapid depressurization of the CO₂ across the valve causes rapid cooling, and coalesces the target compound from the target compound laden supercritical carbon dioxide through expansion cooling, collecting the target compound in a collector vessel (shown as two collector vessels in FIG. 1, and producing carbon dioxide gas.

The gaseous carbon dioxide then flows through a set of coalescing/impingement devices to remove all compounds, and continues through one or more molecular sieves, e.g., 3 Angstrom zeolite molecular sieve media, which removes all material from the gaseous CO₂ (e.g., other than H₂ and He). This cleaned gas flows through a gas booster pump and back to the first heat exchanging device, where it is cooled and re-liquefied; to do this, the supply to the first heat exchanging device and the high intensity optimization pump system is switched from carbon dioxide source container to the collector vessel.

It should be readily understood by the ordinarily skilled artisan in light of the disclosure provided herein that a high intensity targeting (HIT) supercritical fluid extraction system constructed in accordance with the present invention can be manufactured in a variety of shapes and sizes (e.g., with respect container size, connections that service to interconnect and relate multiple components, and equipment or component size/capacity). System components may be formed from metals such as steel, stainless steel (e.g., austenitic and precipitation hardening), aluminum and its alloys, stellite or other superalloys, high nickel alloys such as Inconel, titanium and its alloys, copper beryllium, magnesium, carbon composites, other aramid composites, fiber reinforced polymers, ceramic compounds, fiber reinforced ceramics, as well as other suitable materials, or combinations thereof. Moreover, in certain embodiments of the present invention, and by no means intended to limit the HIT supercritical fluid extraction systems of the present invention in terms of design or construction, e.g., system component composition, the system is stainless steel. In certain embodiments, the system, and components thereof, may comprise materials, e.g., alloys, such that they can maintain ASME B&PVC Section VIII DIV 3 requirements and are capable of safely maintaining pressures of 1000 bar without being subject to temperature embrittlement or stresses. In particular embodiments, the system, and components thereof, may comprise materials suitable to maintain a steady flow rate of liquid carbon dioxide at pressures of about 300 bar to about 6000 bar, e.g., using pump compression ratios as high as 600:1 of drive air pressure (equivalent to 180:1 of pump inlet to outlet pressure as relates to pumps driven in ways other than pneumatically).

A. Carbon Dioxide Source

The HIT supercritical fluid extraction systems of the present invention comprise a carbon dioxide source container. In certain embodiments, the carbon dioxide source container comprises carbon dioxide. In particular embodiments, the carbon dioxide source container is a vessel containing liquid CO₂, e.g., wherein the vessel is a cylinder, e.g., under a pressure of about 62 bar.

In certain embodiments of the present invention, the HIT supercritical fluid extraction system comprises mass flow monitoring by calculations derived from the pump cyclic rate, displacement, and density at inlet conditions.

In certain embodiments of the present invention, the HIT supercritical fluid extraction system comprises a mass flow meter to measure incoming carbon dioxide, i.e., incoming from the source container to the other components of the system. Such mass flow meter may also measure the carbon dioxide returning to the source container, e.g., recycled carbon dioxide.

B. First Heat Exchanging Device

The HIT supercritical fluid extraction systems of the present invention comprise a first heat exchanging device operationally associated with the carbon dioxide source designed to reduce the temperature of the carbon dioxide to maintain the carbon dioxide as a liquid (e.g., to about −15° C.). The first heat exchanger is supplied with coolant to reduce the CO₂ temperature. This cooling is performed to ensure that the CO₂ remains in a liquid state during pumping, especially at higher cyclic rates of the pump (which allows faster system pressurization).

In certain embodiments, the first heat exchanging device is designed to receive carbon dioxide through a path directly from the carbon dioxide source. In particular embodiments the first heat exchanging device is designed to receive carbon dioxide from the recycling path of the recycled carbon dioxide.

C. High Intensity Optimization Pump System

The HIT supercritical fluid extraction systems of the present invention comprise a high intensity optimization pump system capable of maintaining a steady (e.g., controlled) flow rate of the liquid carbon dioxide at pressures greater than or equal to about 300 bar (e.g., greater than or equal to about 350 bar, e.g., greater than or equal to about 680 bar) resulting in highly pressurized liquid carbon dioxide suitable for achieving high intensity extraction targeting.

The high intensity optimization pump system may operate at, for example, atmospheric pressure (1 bar), where the process may use liquid CO₂ in its non-polar state to separate different target compounds (i.e., remove those which are non-polar or particularly volatile before increasing pressure). However, the pump system (e.g., including multiple pumps) is suitable to generate and maintain a steady flow rate of liquid carbon dioxide at pressures of about 300 bar to about 6000 bar, e.g., using pump compression ratios as high as 600:1 of drive air pressure (equivalent to 180:1 of pump inlet to outlet pressure as relates to pumps driven in ways other than pneumatically).

As designed, the actual flow rate is limited only by equipment size, i.e., the order of magnitude of the system. However, in order to achieve certain advantages of the present invention, the present invention affords the ability to exceed 300 bar, e.g., 350 bar, e.g., 680 bar, at a controlled (or maintained) flow rate. Comparatively, the flow rate for certain commercially available systems is around 10 kg/hour (0.16 kg/minute). In stark contrast, in one embodiment of the present invention, pumps of about 10 hp would produce a solvent mass flow of over 50 kg/minute. In particular, the embodiment of FIG. 1 is suitable for 1 kg/minute at maximum pressure and 10 kg/minute up to 4800 psi. A factory size installation would work at nearly an infinite flow rate (1000 kg/min+), without overall process modification other than multiplying the numbers of components and installing them in parallel.

The compression ratio is directly related to the maximum pressure. For example, if the drive air pressure with these pumps is limited to around 120-150 psi, the maximum outlet pressure is only a function of the CR. Pumps are available off the shelf with ratios as high as 500:1 or 600:1, but a pump with a higher CR may be used. As such, in certain embodiments, the high end pressure target is about 6000 bar.

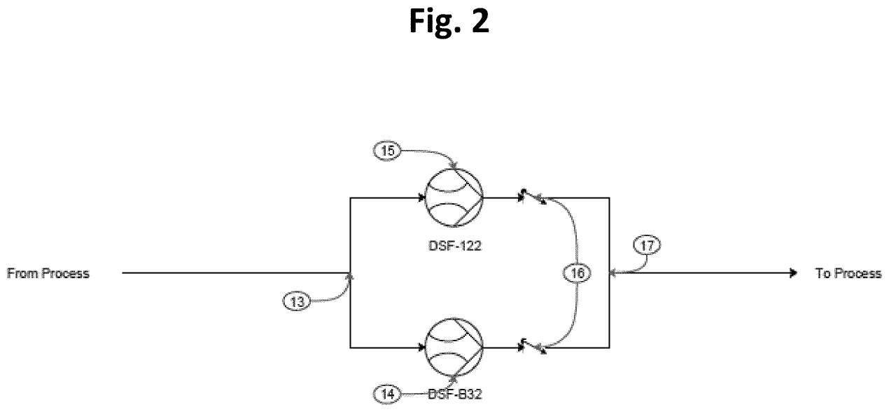

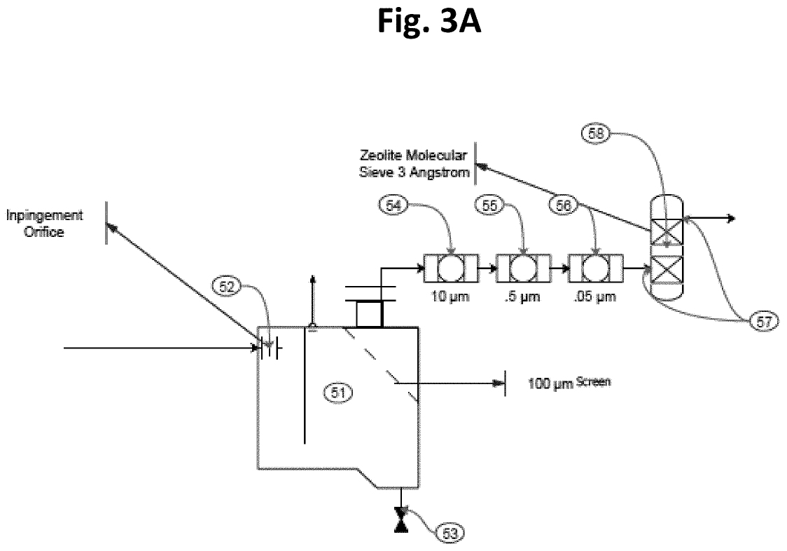

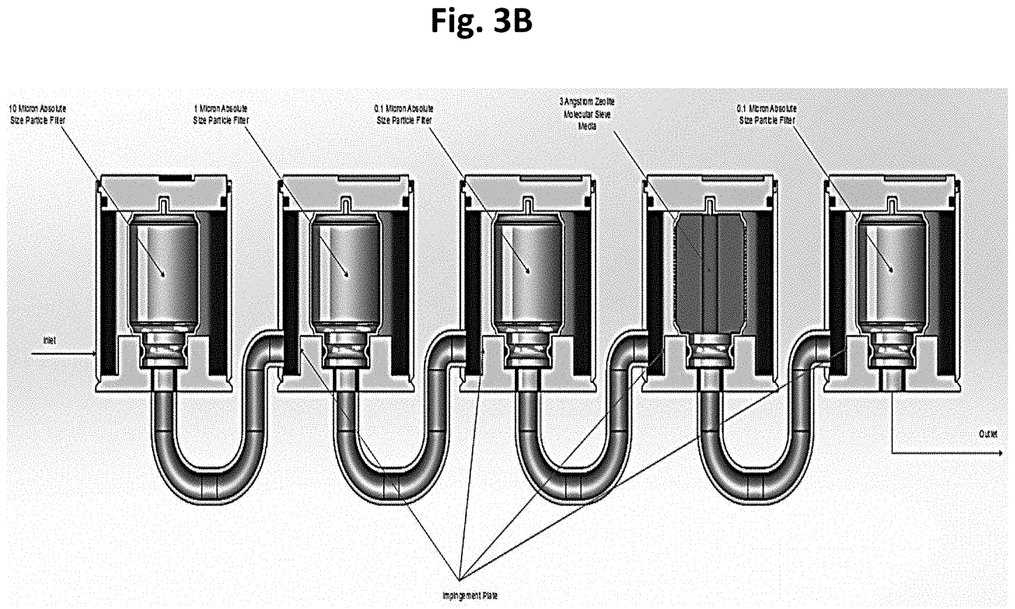

In certain embodiments, the high intensity optimization pump system comprises at least two pumps, further depicted in the schematic of FIG. 2 wherein one pump (e.g., Haskel DSF-B32 29376) has a lower compression ratio, corresponding to a higher flow rate, and the second pump (e.g., Haskel DSF-B122 29376) with a compression ratio capable of reaching ultimate, e.g., pre-defined, pressure that is used throughout the entire pumping process, and is responsible for the addition of CO₂ to the system at pressures above the stall point of the other pump. Such pumps may be air driven piston-type liquid pumps, which, for example, may operate in parallel. In certain embodiments, the high intensity optimization pump system comprises a mid-ratio pump.

D. Second Heat Exchanger Device

The HIT supercritical fluid extraction systems of the present invention comprise a second heat exchanger device operationally associated with the high intensity optimization pump system designed to heat the highly pressurized liquid carbon dioxide to above its critical point, such that supercritical carbon dioxide is formed. In certain embodiments, the second heat exchanger is supplied heated transfer fluid. This heat transfer with the CO₂ increases the temperature to above the critical temperature where the CO₂ is said to be “supercritical”.

In alternative embodiments, the second heat exchanger may utilize direct electrical resistance heating, or induction heating of the process tubing.

E. Extraction Vessel

The HIT supercritical fluid extraction systems of the present invention comprise an extraction vessel designed to hold a pre-determined amount of extractable material, and which is positioned to receive the supercritical carbon dioxide from said second heat exchanger, wherein the extraction vessel may be sealed with the extractable material and supercritical carbon dioxide under highly pressurized conditions to produce a target compound laden supercritical carbon dioxide through high intensity extraction targeting. The size the extraction vessel may vary, and will depend, in certain embodiments, on the amount of extractable material desired to be extracted. In certain embodiments, the size of extraction vessel is greater than 5 L, e.g., 10 L, 20 L, 100 L, or greater.

In certain embodiments of the present invention, the extraction vessel comprises a temperature jacket to maintain specific temperature controls during extraction.

In certain embodiments of the present invention, the HIT supercritical fluid extraction system comprises at least two (e.g., or more) parallel extraction vessels, e.g., operationally associated with at least two collection vessels through the flow regulator device, capable of performing simultaneous or alternating extractions.

In certain embodiments of the present invention, the HIT supercritical fluid extraction system further comprises an extraction vessel agitation mechanism suitable to agitate extraction material for improved extraction of the target compound, e.g., wherein the agitation mechanism is a pump driven recirculating loop.

i. Pin-Retained Closure

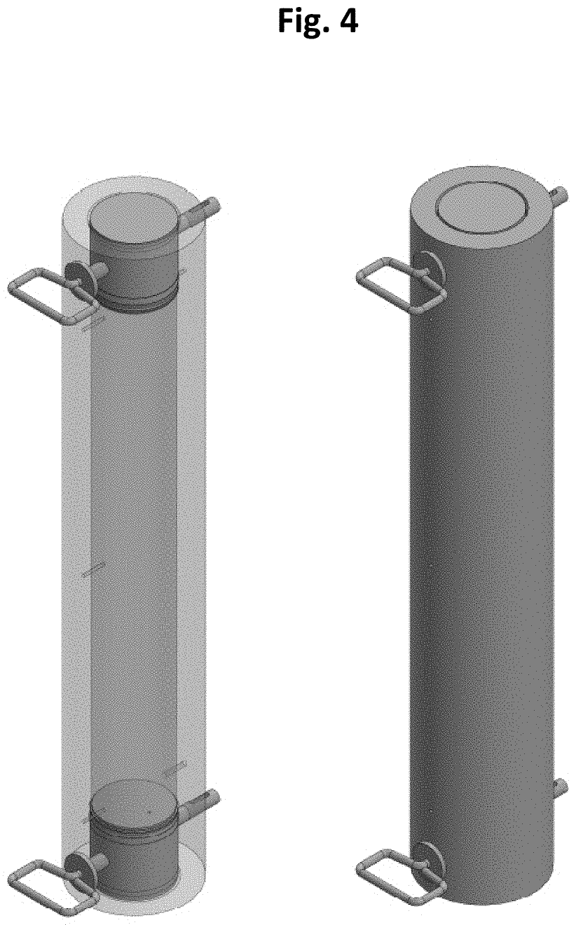

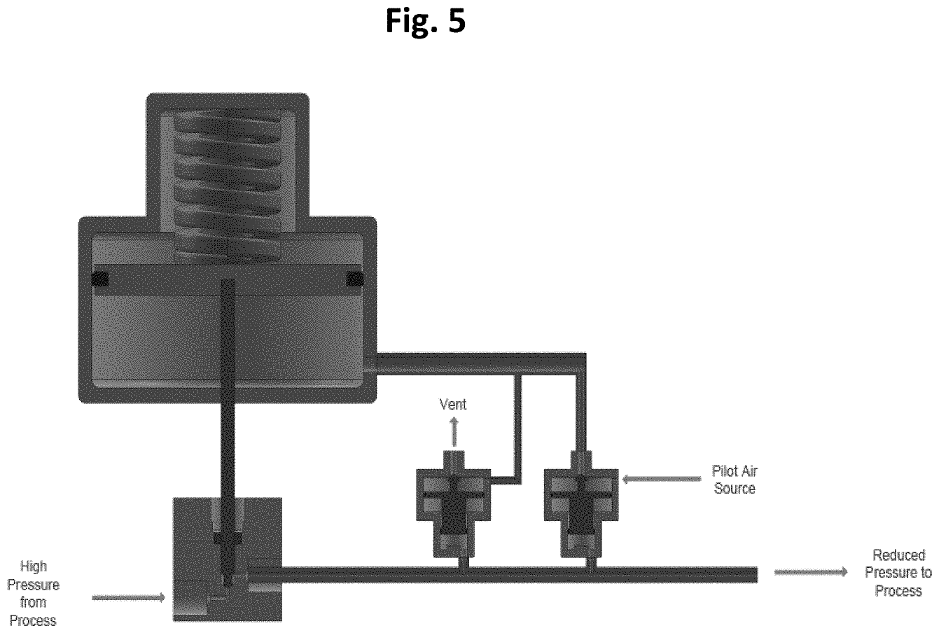

In certain embodiments of the present invention, the extraction vessel comprises a novel pin-retained closure seal, in which a large pin is slid through the vessel walls and the end closure to seal the vessel, and is suitable for high pressure application. This type of closure replaces existing closure designs, such as bolted flange or hammer flange closures. An exemplary embodiment is depicted in FIG. 4.

One advantage of the pin-retained closure seal over the industry standard bolted flange style closure includes the safety improvement characteristic that the vessel cannot be opened once pressure has been applied to the system (even a very small pressure of roughly 2-3 bar) due to the friction applied to the pin. This is a substantial improvement over industry standard closures that can be easily opened while pressurized.

Another advantage of the pin-retained closure seal is that the seals are not subject to over compression during sealing by the operator, which is a common problem with other closure types (e.g., resulting in an operator hammering a threaded closure hard enough to destroy the gasket) The pin-retained closure can be opened and closed in a fraction of the time of other closure types, especially when compared to other closure types capable of sealing at pressures above 500 bar. Further, no tools are required for the operator to open and close the vessel using the pin-retained closure.

Moreover, when compared to most vessels using threaded components io (especially bolted flanges), the lifetime of the pin is nearly infinite. Threads are usually designed to stretch in order to generate their locking force, and have a finite number of cycles in their lifetime. This could easily lead to the use of a fastener which has been weakened over time by an unknowing operator, and a catastrophic system failure in result.

ii. Radial Wedge Ring Modified Bridgman Seal

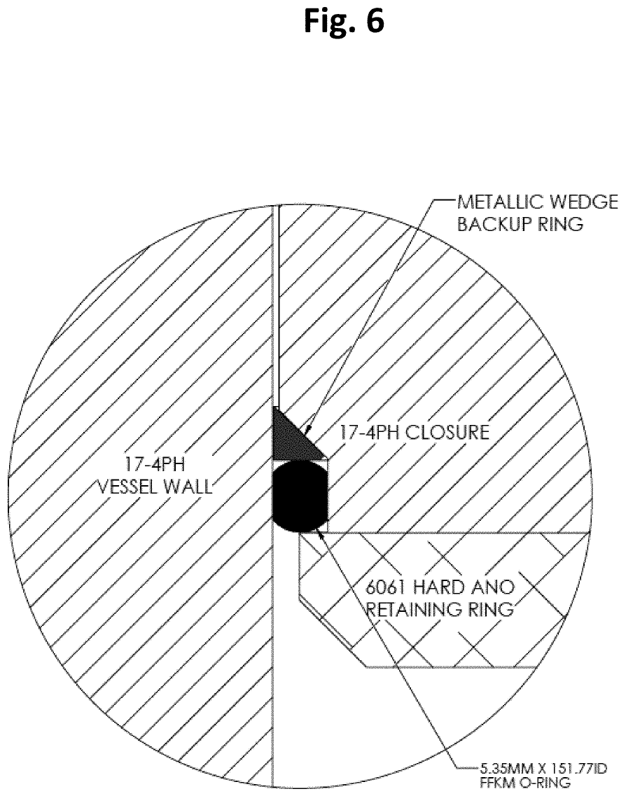

In certain embodiments of the present invention, the extraction vessel comprises a novel radial seal geometry of an advanced Bridgman self-energizing seal (i.e., seals more at higher pressures), which utilizes an expanding hard backup ring behind an elastomeric O-ring for the dual purpose of preventing extrusion of the O-ring at low pressures, and forming a self-energizing seal at pressures above the effective range of the elastomer. FIG. 6 depicts a radial seal of the present invention which is comprised of only one major closure member (as opposed to two in the standard Bridgman seal) and which is self-energizing from the initial seal created by O-ring compression from forced interference. The seal operates under the principle that as pressure is applied in the vessel, the force acting upon the O-ring causes it to push against the wedge ring, the wall of the vessel, and the closure, creating a stronger seal. The O-ring is prevented from extruding into the unsupported space between the closure and wall by the wedge ring, which deforms as more force is applied to expand and seal against the vessel wall and closure taper.

The seal geometry described is a significant advancement over other seal geometries in that it does not require any positive action on behalf of the operator to create the initial seal, and as such is not subject to error in installation. Furthermore, the ease of inserting and removing the closure makes for a reliable high pressure closure which is essentially “self-sealing” to nearly infinite pressure. Moreover, the reduction of components from the original Bridgman seal design allows for substantial cost savings in production. Additionally, the design described herein does not require a step or other contoured feature to be machined in the vessel wall, nor does it require precise machining of a two-part closure as is required by traditional high pressure seals, allowing substantial cost savings as well as ease of maintenance/reconditioning and replacement of components.

F Flow Regulator Device

The HIT supercritical fluid extraction systems of the present invention comprise a flow regulator device suitable for releasing the pressurized target compound laden supercritical carbon dioxide from the extraction vessel in a controlled manner. Such flow regulator devices ensure that downstream pressures do not exceed defined tolerances.

Certain embodiments of the flow regulator device comprise a back pressure regulator (BPR), a device that maintains a defined pressure upstream of itself (at its own inlet). When fluid pressure exceeds the set point the valve opens more to relieve the excess pressure. Back pressure regulators work similarly to relief valves, but the emphasis is on steady state pressure control instead of on/off actuation. The use of a BPR is designed to control upstream pressure, and is still adjusted to ensure that downstream pressures do not exceed tolerance.

i. Micro Metering Expansion Valve

Given that BPRs are challenging to construct for high pressures (over 680 bar) and must usually be custom built at high cost, in certain embodiments of the present invention the flow regulator device is a micro metering expansion valve. This micro-metering valve may be throttled using air pressure which is modulated (either electronically or mechanically) by the downstream process fluid pressure to maintain a specified pressure in the collector vessel (e.g., about 35 bar).

BPRs are also highly liable to freeze, with membrane failure being a common issue. Due to their large size, and the distance from the exterior to the point of fluid expansion, they are difficult to heat to avoid this problem, often resulting in overheating io of inlet and outlet fluids. BPRs are also highly liable to clog due to particulate presence.

In particular embodiments of the present invention, the micro-metering valve is a needle valve, e.g., an air actuated needle valve with a micro-metering stem design. The actuator is of a normally closed design in which a heavy spring presses the stem against its seat. Air pressure is introduced to the actuator body to counteract this spring force. By precisely controlling the pressure in the actuator, the distance which the valve opens can be accurately and quickly adjusted to permit the correct flow. Two different methods, i.e., electronically or mechanically, have been utilized, and each embodiment separately uniquely supplies air pressure to the actuator.

In the first embodiment, the pressure is read downstream of the valve by a transducer. When the pressure approaches a setpoint, a logic control device interfaces with a solenoid proportional valve to reduce the air pressure slightly based on the velocity of the pressure rise. For example, in certain embodiments, this may be conducted using a PID controller (proportional-integral-derivative controller), e.g., which would be commonly used for heater control. When the setpoint pressure is approached, the valve will drop the actuator pressure, closing the valve. By tuning the rate of response of the valve, the air pressure in the actuator can be balanced at a level above the pressure required to crack the valve open with the added force placed on the needle by the process fluid, but below the pressure required to open the valve with no process fluid force. At this pressure, the size of the orifice created in the valve is balanced by the pressure differential, causing the stem to “float”. The differential pressure can be adjusted as needed by adjusting the actuator pilot pressure. The use of a needle valve in lieu of a BPR allows operation at high pressures at substantially decreased cost. Heating of the valve is easier, and there is less risk of failure due to icing. Due to the hard seats and high closing pressure, particulate contamination is less likely to “hang up” the valve, and is more easily cleared. Additionally, the cost of valves for large systems (process pipes ½″ and up) can be orders of magnitude less than those using BPRs.

In the second embodiment, a ratio BPR is piloted by the downstream pressure and used to adjust the supply of air pressure to the micro-metering valve actuator. As the downstream system pressure approaches the max allowed pressure, the BPR restricts the supply of air pressure to the actuator. When the max pressure is met, the BPR is sealed by the process pressure, shutting off pressure to the actuator. This system has the advantage of substantially faster response time, but is less adjustable. It still solves the aforementioned problems with a BPR as a flow regulating device, despite using a BPR. The BPR used to regulate the flow into the initial BPR is a smaller, off-the-shelf component and does not have to contend with particulate matter or rapid expansion cooling effects.

G. Collector Vessel

The HIT supercritical fluid extraction systems of the present invention comprise a collector vessel designed to coalesce the target compound from the target compound laden supercritical carbon dioxide through expansion cooling, collecting the target compound and producing carbon dioxide gas. In certain embodiments, the size of collection vessel is greater than 1 L, e.g., greater than 5 L, e.g., 10 L, 20 L, 50 L, or greater. For example, factory level installations could be on the order of 20,000 liters

In certain embodiments, the HIT supercritical fluid extraction systems of the present invention comprise a collector vessel which is designed to cause cyclonic flow of gases in order to develop a pressure differential within the vessel to ensure that only the less dense, unladen CO₂ gas is exhausted or recovered.

In certain embodiments of the present invention, the HIT supercritical fluid extraction system further comprises a fractionating collection system. For example, the system may be adapted to fractionate target compounds precisely with minimal loss using large column supercritical fluid chromatography.

H. Additional Components

Moreover, in certain embodiments, the HIT supercritical fluid extraction systems of the present invention may incorporate additional design elements or components that do not significantly inhibit or prevent the features of the HIT supercritical fluid extraction systems explicitly described herein. Such additional design elements may be for the sole purpose of aesthetic design, or for utility purposes, e.g., coalescing, metering pumps, or agitation.

i. Coalescing And Impingement Devices

In certain embodiments of the present invention, the HIT supercritical fluid extraction system further comprises one or more coalescing and impingement devices. In certain embodiments of the present invention, the HIT supercritical fluid extraction system further comprises a molecular sieve. In particular embodiments, the molecular sieve is a 3 angstrom molecular sieve. The use of 3 angstrom molecular sieve (which is only slightly larger than CO₂ at 2.8 A) is a method used to purify CO₂ for use as an analytical solvent. By using this filter media for the solvent recapture process, the CO₂ can be maintained at analytical solvent levels of purity, and as such can be used as a solvent source for supercritical fluid chromatography.

The use of a molecular sieve to purify solvent supply is a new and highly beneficial process of the present invention. A common issue with all botanical extractors is contamination from one batch to the next. In particular, light, nonpolar hydrocarbons are extremely difficult to remove from solution with CO₂. Many of these contaminants (hydrocarbons and pesticides) can pose severe health and process problems, especially as they become more concentrated over the life of the solvent supply. By using a 3 A molecular sieve, the compounds can be removed completely from the solvent, e.g., and then recycled.

In certain embodiments of the present invention, the HIT supercritical fluid extraction system further comprises a gas pump system designed to direct the carbon dioxide gas from the collector vessel to the first heat exchanging device to recycle the carbon dioxide gas, producing recycled liquid carbon dioxide. By using the existing liquid pump(s) to recycle the solvent into the carbon dioxide source container, several advantages are afforded:
The pressure in the collector vessels can be substantially lower, improving the removal of the target compounds from the solution as well as reducing the cost of those vessels. This is because the gas pump does not need to make up as much of a pressure difference to return the solvent to a liquid phase.
Due to the lower collector vessel pressure, the gas pump does not need to remove as much solvent from the collector vessels as it would otherwise, allowing a smaller, less expensive pump to be used for the same throughput.
By reducing the outlet pressure required of the gas booster pump (34 bar vs 62 bar), the pump ratio allows the pump to operate below atmospheric pressure. This is a major advantage from a cost standpoint as it means that only the CO₂ which is dissolved in the extracted and coalesced material is lost in each cycle, equating to at least a 90% reduction in solvent use relative to industry standards. This also decreases the amount of CO₂ which is reintroduced into the atmosphere. Additionally, operator safety is drastically improved as standard systems can easily allow buildup of CO₂ in the room to above safe limits (they usually maintain 14 bar-55 bar of pressure). The residual pressure in standard systems also poses an occupational safety risk in that several thousand pounds of force may still be applied to the pressure vessel closure when the operator removes it, and the safe letdown of that pressure is controlled only by the judgement of the operator.

In certain embodiments of the present invention, the HIT supercritical fluid extraction system further comprises a valve that is suitable for directing the recycled liquid carbon dioxide to the carbon dioxide source vessel.

In certain embodiments of the present invention, the molecular sieve may be used as a self-cleaning cycle that may be run on the machine in which solvent is cycled through the machine until all contamination has been captured by the filter media, saving substantial time and cost in cleaning (as well as improving safety by limiting the requirement for fitting breakdown).

In certain embodiments of the present invention, contaminants can be specifically targeted for dissolution from otherwise unusable material and then removed from the solvent by the filter media, and thereby purifying the material.

In certain embodiments of the present invention, the system can be recharged with utility-grade CO₂ which will be purified to analytical grade prior to use. This potentially reduces the cost of operation drastically.

ii. Metering Pump

In certain embodiments of the present invention, the HIT supercritical fluid extraction system further comprises a cosolvent pump. In certain embodiments, a metering pump may be used to introduce a ratio of additional solvent, e.g., polar solvent (such as ethanol), to the CO₂ supply prior to chilling, for example to increase the rate and efficiency of dissolution of certain target compounds, and which may also improve selectivity of dissolution.

iii. Parallel Pathways

In certain embodiments of the present invention, the HIT supercritical fluid extraction system comprises two or more parallel sets of extraction and collector vessels to provide efficient extractable material processing, wherein such parallel sets may be alternatively emptied and reloaded while the other is being processed. This may be reduced to a single vessel (for lower cost), or increased to any number of vessels.

iv. Agitation

In certain embodiments of the present invention, the HIT supercritical fluid extraction system further comprises an agitation mechanism suitable to agitate the material being extracted. In certain embodiments, the agitation mechanism is a recirculating loop positioned off the extraction vessels. This allows a pump which is much smaller and less expensive than the liquid pumps being used to pressurize the system to be used to mix solvent within the vessel. By agitating the solvent, more unladen solvent will be brought into contact with the solute, and bulk transfer of solute in the solvent volume is improved. This recirculating loop may also be installed with a heat exchanger, which can be used to maintain or change the temperature of the contents of the vessels. This is a substantial improvement over designs which use a jacketed vessel because with jacketed vessels, the wall thickness of the vessels do not allow rapid temperature changes, and the energy required to heat or cool the vessel is substantially higher due to the thermal mass of the vessel itself; and further the extraction vessel design requirements would be more severe due to the extreme temperatures that are required in the jackets.

v. Control Interface

In certain embodiments of the present invention, the HIT supercritical fluid extraction system further comprises a control interface. In certain embodiments, the HIT supercritical fluid extraction system comprise a safety logic control interface, e.g., a PLC—Programmable Logic Controller. In specific embodiments the interface provides automated capability.

Such controlling interfaces afford safety protocols using both hardware and software logic protocols through redundant processing, to ensure that in the case of failure of one processor, an unsafe condition cannot be created. In fact, safety protocol is required by many codes and jurisdictions for any hardware which relies on PLCs for essential safety functions; however, current systems do not offer such safety enhancements.

vi. Chromatography

In certain embodiments of the present invention, the HIT supercritical fluid extraction system further comprises a chromatographic column. In certain embodiments, the HIT supercritical fluid extraction system further adapts the system with an extra port in the extraction vessel to connect to a high precision regulator, a sample loading chamber, and a chromatography column, e.g., in sequence.

In certain embodiments, the user may first run a blank sample of CO₂ through the chromatograph prior to loading material in the extraction vessel to test the calibration of the detector (e.g., mass spectrometer or inductively coupled plasma detector) as well as to certify the purity of the solvent. This blank run may also be useful to determine a need to replace the molecular sieve material. Another run with analytical testing standards may be undertaken to further calibrate the detector. From this point, samples may be placed in the sample loading chamber and tested as they would with a standalone chromatograph. Alternatively, the chamber may be loaded with material and brought to pressure, and a sample could be run through the chromatograph prior to depressurizing the vessel. It is worth noting that supercritical fluid chromatography is superior to high performance liquid chromatography in nearly every respect, but is normally a price-limited technology due to the cost of providing a solvent source of supercritical CO₂.

Additionally, UHPLC (ultra-high performance liquid chromatography) levels of resolution can be achieved with supercritical fluid (SCF), but pressures of around 1000 bar are required to do so. In this regard, present system is unique as no existing system can produce sufficient pressure to meet this requirement.

III. Methods of the Invention

Systems of the present invention are provided herein as certain exemplary embodiments of implementation of the novel methods of the present invention. Methodologies and steps described above in relation to the systems of the present invention are therefore intended to be incorporated further herein as exemplary methods; and form part of the embodiments described specifically in the methods of the invention by reference hereto.

A. Methods Of High Intensity Targeting (HIT) Supercritical Fluid Extraction

Another embodiment of the present invention provides a method of high intensity targeting (HIT) supercritical fluid extraction comprising the steps of
providing carbon dioxide from a source container (e.g., comprising carbon dioxide, e.g., a vessel containing liquid CO₂);
reducing the temperature of the carbon dioxide to maintain the carbon dioxide as a liquid (e.g., to about −15° C.), e.g., using a first heat exchanging device operationally associated with the carbon dioxide source container; e.g., designed to receive carbon dioxide through a path directly from the carbon dioxide source, e.g., and further from the recycling path;
pumping the carbon dioxide liquid to result in highly pressurized liquid carbon dioxide suitable for achieving high intensity extraction targeting by increasing the flow of the carbon dioxide, and maintaining a steady (e.g., controlled) flow rate of the liquid carbon dioxide at pressures greater than or equal to about 300 bar (e.g., greater than or equal to about 350 bar, e.g., greater than or equal to about 680 bar);
heating the highly pressurized liquid carbon dioxide to above its critical point, such that supercritical carbon dioxide is formed, e.g., using a second heat exchanger device operationally associated with the high intensity optimization pump system;
sealing an extractable material (e.g., a pre-determined amount of extractable material) with the supercritical carbon dioxide under pressurized conditions to produce a target compound laden supercritical carbon dioxide through high intensity extraction targeting (e.g., wherein the extraction vessel comprises a pin-retained closure seal), e.g., using an extraction vessel designed to hold a pre-determined amount of extractable material, and which is positioned to receive the supercritical carbon dioxide from said second heat exchanger;
releasing the pressurized target compound laden supercritical carbon dioxide from the extraction vessel in a controlled manner by flow regulation, e.g., using a flow regulator device, e.g., a micro metering expansion valve; and
collecting the target compound and producing carbon dioxide gas by coalescing the target compound from the target compound laden supercritical carbon dioxide through expansion cooling,

such that high intensity supercritical fluid extraction of a target compound is achieved. In certain embodiments of the methods of the present invention, the method further comprises the post-processing step of using a centrifuge to separate target compounds from unwanted compounds, e.g., prior to other post-processing methods. In certain embodiments, specific conditions may be modified to improve extraction, including: temperature, pressure, flow, and time.

Yet another embodiment of the present invention provides a method of high intensity targeting (HIT) supercritical fluid dual cycle extraction comprising a first cycle extraction method at low temperature, and relatively low pressure, e.g., trans-critical (e.g., at ˜20 C and ˜75 bar), e.g., to extract the desirable, nonpolar, volatile terpene compounds; and second cycle extraction utilizing a high intensity targeting (HIT) supercritical fluid extraction of the present invention. In certain embodiments, the products of the two steps would be coalesced in a different collector vessels, e.g., so that they could be processed separately.

In certain embodiments of the present invention, the method of HIT supercritical fluid extraction further comprises the step of recycling the carbon dioxide gas by reducing the temperature of the carbon dioxide gas after collection of the target compound, e.g., by directing the carbon dioxide gas from the collector vessel to the first heat exchanging device, to recycle the carbon dioxide gas by producing recycled liquid carbon dioxide, e.g., using a gas pump system.

In certain embodiments of the present invention, the method of HIT supercritical fluid extraction further comprises the step of filtration of the recycled carbon dioxide gas using one or more coalescing and impingement devices.

In certain embodiments of the present invention, the method of HIT supercritical fluid extraction further comprises the step of filtration of the recycled carbon dioxide gas using one or more molecular sieves. In particular embodiments, the molecular sieve is a 3 angstrom molecular sieve. The use of 3 angstrom molecular sieve (which is only slightly larger than CO₂ at 2.8 A) is a method used to purify CO₂ for use as an analytical solvent. By using this filter media for the solvent recapture process, the CO₂ can be maintained at analytical solvent levels of purity, and as such can be used as a solvent source for supercritical fluid chromatography.

In certain embodiments of the present invention, the method comprises the step of removing CO₂ from the collector vessel(s), e.g., by running the liquid pumps. In certain embodiments, the outlet liquid CO₂ is first directed through the high-pressure section of the system to “wash down” any target compounds which were dissolved but precipitated in the extraction vessel. Further, once a sufficient amount of CO₂ is cycled through the system in the wash down, the outlet of the liquid pumps switches to a check valve which is used to recycle the CO₂ back into the source container. As the CO₂ is replaced in the source container, the extraction vessel(s)' pressure will fall back down to the collector vessel target pressure. As the pressure falls below the collector vessel target pressure, the gas booster pump is run to evacuate all solvent down to atmospheric pressure in all extraction vessels and to ensure that gas pressure to the chiller first heat exchanging device is sufficient to achieve liquid phase condition so that it can be pumped back into storage. Once the system is fully depressurized, the extraction vessels may be opened by the operator so that the depleted organic material and separated target compounds may be removed.

In certain embodiments of the present invention, the method of HIT supercritical fluid extraction the method is automated. In particular embodiments, the automation may comprise automated logic control of one or more of the valves, pumps, heaters, chillers, etc of the systems of the present invention.

In certain embodiments of the methods of the present invention, the extraction period is substantially reduced compared to systems that are not capable of pumping highly pressurized liquid carbon dioxide in a manner suitable for achieving high intensity extraction targeting, e.g., about 50% or greater reduction of the extraction period, e.g., e.g., about 60% or greater reduction of the extraction period, e.g., about 70% or greater reduction of the extraction period, e.g., about 80% or greater reduction of the extraction period, e.g., about 90% or greater reduction of the extraction period, e.g., about 95% or greater reduction of the extraction period. In particular embodiments, the extraction period is reduced from about 10 hours per run to about 1. In particular embodiments, the extraction period is reduced from about 3 hours per run to about 1.

Extraction of specific compounds, in certain embodiments of the present invention, may be considered to be fractionation using solubility or dissolution (as opposed to boiling point/vapor pressure for thermal fractionation). In particular embodiments, this process is useful in scenarios such as biosynthesis post-processing where the target compounds and other process compounds have differing polarities or solubility in supercritical CO₂ (SCO₂), but similar boiling points. Thus, separating them using the tunable solvating characteristics in SCO₂ would be faster/easier/more effective than thermal fractionation/distillation. Moreover, with respect to cannabis, supercritical fluid fractionation using HIT would be highly beneficial given that thermal distillation/fractionation carries a higher level of loss, especially because many of the target compounds have very similar boiling points.

In certain embodiments of the present invention, the methods (and systems) of the present invention may be used to perform multi-step purification of final extracted target compounds, using a first extraction that would be performed under high intensity conditions (e.g., 1000 bar) described herein, to extract nearly all of the available target compounds from the extractable material matrix. In a second processing/purification step, the methods and systems may be used to reprocess the extracted target material at transcritical conditions during a second run. This second run would dissolve nearly all of the target compounds extracted in the first run (because high dissolution forces would no longer be needed to remove the compounds from the plant material matrix), but the unwanted compounds from the first run would be insoluble and left out of the second run yield.

In certain embodiments of the present invention, the invention provides a method comprising the step of reintroduction of a target compound into one of the extraction vessels and the use a very high-pressure differential orifice in a collector vessel to enact rapid expansion of supercritical solution (RESS), which forms very fine precipitates which are easily pressed into pill form. Although this is the process which large pharmaceutical companies use to produce consistent medical products (e.g., ibuprofen), it is usually limited to very large production scales due to the cost of creating a supply of supercritical C₂; however, the system and methods described herein overcome this limitation, e.g., capable in a much smaller scale, e.g., in a cost effective manner.

A. Providing Carbon Dioxide

In certain embodiments of the present invention, the method of high intensity targeting (HIT) supercritical fluid extraction comprises the step of providing carbon dioxide from a source container. In certain embodiments, the carbon dioxide source container comprises carbon dioxide. In particular embodiments, the carbon dioxide source container is a vessel containing liquid CO₂, e.g., wherein the vessel is a cylinder, e.g., under a pressure of about 62 bar.

B. Reducing Temperature

In certain embodiments of the present invention, the method of high intensity targeting (HIT) supercritical fluid extraction comprises the step of reducing the temperature of the carbon dioxide to maintain the carbon dioxide as a liquid (e.g., to about −15° C.). In certain embodiments, the first heat exchanger is supplied with coolant to reduce the CO₂ temperature. This cooling is performed to ensure that the CO₂ remains in a liquid state during pumping, especially at higher cyclic rates of the pump (which allows faster system pressurization).

In an alternative embodiment, the first heat exchanger cooling may be performed using heat exchange from expansion valve expansion-cooling.

In an alternative embodiment, the first heat exchanger cooling may be performed using Peltier-effect cooling.

In an alternative embodiment, the first heat exchanger cooling may be performed using heat exchange from direct refrigeration heat exchange with the process fluid.

C. Pumping Carbon Dioxide Liquid

In certain embodiments of the present invention, the method of high intensity targeting (HIT) supercritical fluid extraction comprises the step of pumping the carbon dioxide liquid to result in highly pressurized liquid carbon dioxide suitable for achieving high intensity extraction targeting by increasing the flow of the carbon dioxide, and maintaining a steady (e.g., controlled) flow rate of the liquid carbon dioxide at pressures greater than or equal to about 300 bar (e.g., greater than or equal to about 350 bar, e.g., greater than or equal to about 680 bar) resulting in highly pressurized liquid carbon dioxide suitable for achieving high intensity extraction targeting.

The methods of high intensity targeting using supercritical fluid extraction may comprise the step of pumping (e.g., using multiple pumps) a steady flow rate of liquid carbon dioxide at pressures of about 300 bar to about 6000 bar, e.g., using pump compression ratios as high as 600:1 of drive air pressure (equivalent to 180:1 of pump inlet to outlet pressure as relates to pumps driven in ways other than pneumatically).

Comparatively, the flow rate for certain commercially available systems is around 10 kg/hour (0.16 kg/minute). In stark contrast, in one embodiment of the present invention, pumps of about 10 hp would produce a solvent mass flow of over 50 kg/minute. In particular, the embodiment of FIG. 1 is suitable for 1 kg/minute at maximum pressure and 10 kg/minute up to 4800 psi. A factory size installation would work at nearly an infinite flow rate (1000 kg/min+), without overall process modification other than multiplying the numbers of components and installing them in parallel.

D. Heating Highly Pressurized Liquid Carbon Dioxide

In certain embodiments of the present invention, the method of high intensity targeting (HIT) supercritical fluid extraction comprises the step of heating the highly pressurized liquid carbon dioxide to above its critical point, such that supercritical carbon dioxide is formed, e.g., using a second heat exchanger device operationally associated with the high intensity optimization pump system. The second heat exchanger is supplied heated transfer fluid. This heat transfer with the CO₂ increases the temperature to above the critical temperature where the CO₂ is said to be “supercritical”.

In alternative embodiments, the method of high intensity targeting (HIT) supercritical fluid extraction comprises the step of heating the highly pressurized liquid carbon dioxide to above its critical point may utilize direct electrical resistance heating, or induction heating of the process tubing.

E. Sealing Extractable Material Under Pressure

In certain embodiments of the present invention, the method of high intensity targeting (HIT) supercritical fluid extraction comprises the step of sealing an extractable material (e.g., a pre-determined amount of extractable material) with the supercritical carbon dioxide under pressurized conditions to produce a target compound laden supercritical carbon dioxide through high intensity extraction targeting (e.g., wherein the extraction vessel comprises a pin-retained closure seal), e.g., using an extraction vessel designed to hold a pre-determined amount of extractable material, and which is positioned to receive the supercritical carbon dioxide from said second heat exchanger. The size the extraction vessel may vary, and will depend, in certain embodiments, on the amount of extractable material desired to be extracted. In certain embodiments, the size of extraction vessel is greater than 5 L, e.g., 10 L, 20 L, 100 L, or greater.

In certain embodiments of the present invention, the extraction vessel temperature may be controlled, e.g., using a temperature jacket or heated agitation to maintain specific temperature controls during extraction.

In certain embodiments of the present invention, the extraction may be performed using at least two parallel extraction vessels, capable of performing simultaneous or alternating extractions.

F. Releasing Pressurized Target Compound Laden Supercritical Carbon Dioxide

In certain embodiments of the present invention, the method of high intensity targeting (HIT) supercritical fluid extraction comprises the step of releasing the pressurized target compound laden supercritical carbon dioxide from the extraction vessel in a controlled manner by flow regulation, e.g., using a flow regulator device, e.g., a micro metering expansion valve.

G. Collecting Target Compound

In certain embodiments of the present invention, the method of high intensity targeting (HIT) supercritical fluid extraction comprises the step of collecting the target compound and producing carbon dioxide gas by coalescing the target compound from the target compound laden supercritical carbon dioxide through expansion cooling. In certain embodiments, the size of collection vessel is greater than 1 L, e.g., greater than 5 L, e.g., 10 L 20 L, 50 L or greater.

In certain embodiments of the present invention, the coalescing is performed in part by a collector vessel which is designed to cause cyclonic flow of gases in order to develop a pressure differential within the vessel to ensure that only the less dense, unladen CO₂ gas is exhausted or recovered.

In certain embodiments of the present invention, the method further comprises fractionated collection. For example, the system may be adapted to fractionate target compounds precisely with minimal loss using large column supercritical fluid chromatography.

### EXEMPLIFICATION

Having thus described the invention in general terms, reference will now be made to the accompanying drawings of exemplary embodiments, which are not necessarily drawn to scale, and which are not intended to be limiting in any way.

In this respect, it is to be understood that the invention is not limited in its application to the details of construction and to the arrangements of the components set forth in the following description or illustrated in the Figures. The invention is capable of other embodiments and of being practiced and carried out in various ways. Also, it is to be understood that the phraseology and terminology employed herein are for the purpose of description and should not be regarded as limiting.

### Example 1

### An Exemplary High Intensity Targeting (HIT) Supercritical Fluid Extraction System

FIG. 1 depicts a schematic view of a high intensity targeting (HIT) supercritical fluid extraction system of certain embodiments of the present invention. The carbon dioxide source container provides liquid carbon dioxide to the first medium pressure tube-in-tube style or brazed-plate style heat exchanger device operationally associated with the carbon dioxide source designed to reduce the temperature of the carbon dioxide to maintain the carbon dioxide as a liquid. The high intensity optimization pump system comprising a DSF-122 air-driven positive displacement liquid pump with a 122:1 compression ratio manufactured by Haskel, which is suitable for maintaining a steady flow rate of the liquid carbon dioxide at pressures greater than or equal to about 1000 bar, and a DSF-B32 air driven positive displacement liquid pump with a 32:1 compression ratio is suitable for maintaining a steady flow rate of the liquid carbon dioxide at pressures greater than or equal to about 300 bar resulting in highly pressurized liquid carbon dioxide suitable for achieving high intensity extraction targeting. Once the liquid carbon dioxide is highly pressurized, the second heat exchanger device, which may be a tube-in-tube heat exchanger, e.g., manufactured by Excergy, operationally associated with an immersion electric resistance heater manufactured by Omega Instruments that is io operationally associated with the high intensity optimization pump system, is used to heat the highly pressurized liquid carbon dioxide to above its critical point, such that supercritical carbon dioxide is formed.

The supercritical carbon dioxide is then received in a 20 liter high pressure extraction vessel manufactured from 17-4PH (fitted with pin-retained closure seals and radial wedge ring seal described herein) from said second heat exchanger, which is designed to hold a pre-determined amount of extractable material. The extraction vessel is fitted with a platinum resistance temperature transmitter manufactured by Noshok, and a pressure transducer manufactured by Noshok to help monitor the extraction process and to ensure production of a target compound laden supercritical carbon dioxide through high intensity extraction targeting.

The flow regulator device suitable for releasing the pressurized target compound-laden supercritical carbon dioxide from the extraction vessel in a controlled manner is a micro metering expansion valve, for example a wide-pattern stem air-actuated needle valve manufactured by Haskel Butech. In certain embodiments, the flow regulator device is piloted by an ITV00 electronic pressure regulator manufactured by SMC.

The target compound is then coalesced from the target compound laden supercritical carbon dioxide through expansion cooling in a 15 liter vessel with the same aforementioned seal geometry and closure type as the extraction vessel capable of acting as a cyclonic coalescing device, and producing carbon dioxide gas. The system comprises two parallel extraction vessels operationally associated with at least two collection vessels through the flow regulator device capable of performing simultaneous or alternating extractions.

A gas pump system is shown using an AGD-4 air-driven gas booster manufactured by Haskel designed to direct the carbon dioxide gas from the collector vessel to the first heat exchanging device to recycle the carbon dioxide gas, producing recycled liquid carbon dioxide. Moreover the system further comprises a valve that is suitable for directing the recycled liquid carbon dioxide to the carbon dioxide source vessel.

Additional coalescing and impingement devices are used to for filtration of the carbon dioxide gas comprising a 1 liter breech locking pressure vessel serving as an impingement device and housing filter elements. These filter elements include:
A 10 micron absolute-particle-size stainless steel filter element manufactured by Hydac GMBH;
A 1 micron absolute-particle-size stainless steel filter element manufactured by Hydac GMBH;
A 0.1 micron absolute-particle-size stainless steel filter element manufactured by Hydac GMBH; and
A 3 Angstrom zeolite molecular sieve media manufactured by 3M;

The system further comprises a control interface, selected as a S7-1515F programmable logic controller (PLC) manufactured by Siemens, and a TP-1500 human-machine interface (HMI) manufactured by Siemens. These interfaces serve to provide a certain level of automated capability.

### INCORPORATION BY REFERENCE

The entire contents of all patents, published patent applications and other references cited herein are hereby expressly incorporated herein in their entireties by reference.

### EQUIVALENTS

Those skilled in the art will recognize, or be able to ascertain using no more than routine experimentation, numerous equivalents to the specific procedures described herein. Such equivalents are considered to be within the scope of this invention and are covered by the following claims. Moreover, any numerical or alphabetical ranges provided herein are intended to include both the upper and lower value of those ranges, unless clearly contradicted explicitly or by the context. In addition, any listing or grouping is intended, at least in one embodiment, to represent a shorthand or convenient manner of listing independent embodiments; as such, each member of the list should be considered a separate embodiment.

What is claimed is:

1. A high intensity targeting (HIT) supercritical fluid extraction system comprising
a carbon dioxide source container;
a first heat exchanging device operationally associated with the carbon dioxide source designed to reduce the temperature of the carbon dioxide to maintain the carbon dioxide as a liquid;
a high intensity optimization pump system capable of maintaining a steady flow rate of the liquid carbon dioxide at pressures greater than or equal to about 300 bar resulting in highly pressurized liquid carbon dioxide suitable for achieving high intensity extraction targeting;
a second heat exchanger device operationally associated with the high intensity optimization pump system designed to heat the highly pressurized liquid carbon dioxide to above its critical point, such that supercritical carbon dioxide is formed;
an extraction vessel designed to hold a pre-determined amount of extractable material, and which is positioned to receive the supercritical carbon dioxide from said second heat exchanger, wherein the extraction vessel may be sealed with the extractable material and supercritical carbon dioxide under highly pressurized conditions to produce a target compound laden supercritical carbon dioxide through high intensity extraction targeting;
a flow regulator device suitable for releasing the pressurized target compound laden supercritical carbon dioxide from the extraction vessel in a controlled manner; and
a collector vessel designed to coalesce the target compound from the target compound laden supercritical carbon dioxide through expansion cooling, collecting the target compound and producing carbon dioxide gas.

2. The HIT supercritical fluid extraction system of claim 1, further comprising a gas pump system designed to direct the carbon dioxide gas from the collector vessel to the first heat exchanging device to recycle the carbon dioxide gas, producing recycled liquid carbon dioxide.

3. The HIT supercritical fluid extraction system of claim 2, further comprising a valve that is suitable for directing the recycled liquid carbon dioxide to the carbon dioxide source vessel.

4. The HIT supercritical fluid extraction system of claim 1, further comprising one or more coalescing and impingement devices.

5. The HIT supercritical fluid extraction system of claim 1, further comprising a molecular sieve.

6. The HIT supercritical fluid extraction system of claim 5, wherein the molecular sieve is a 3 angstrom molecular sieve.

7. The HIT supercritical fluid extraction system of claim 1, wherein the flow regulator device is a micro metering expansion valve.

8. The HIT supercritical fluid extraction system of claim 1, further comprising a control interface.

9. The HIT supercritical fluid extraction system of claim 8, further comprising a safety logic control interface.

10. The HIT supercritical fluid extraction system of claim 9, wherein the interface provides automated capability.

11. The HIT supercritical fluid extraction system of claim 1, wherein the extractable material is a botanical material.

12. The HIT supercritical fluid extraction system of claim 1, wherein the extraction vessel comprises a pin-retained closure seal.

13. The HIT supercritical fluid extraction system of claim 1, further comprising a cosolvent pump.

14. The HIT supercritical fluid extraction system of claim 1, wherein the extraction vessel comprises a temperature jacket to maintain specific temperature controls during extraction.

15. The HIT supercritical fluid extraction system of claim 1, wherein the system comprises at least two parallel extraction vessels operationally associated with at least two collection vessels through the flow regulator device capable of performing simultaneous or alternating extractions.

16. The HIT supercritical fluid extraction system of claim 1, further comprising an extraction vessel agitation mechanism suitable to agitate extraction material for improved extraction of the target compound.

17. The HIT supercritical fluid extraction system of claim 1, further comprising a chromatographic column.

18. A method of high intensity targeting (HIT) supercritical fluid extraction comprising the steps of
providing carbon dioxide from a source container;
reducing the temperature of the carbon dioxide to maintain the carbon dioxide as a liquid;
pumping the carbon dioxide liquid to result in highly pressurized liquid carbon dioxide suitable for achieving high intensity extraction targeting by increasing the flow of the carbon dioxide, and maintaining a steady flow rate of the liquid carbon dioxide at pressures greater than or equal to about 300 bar;
heating the highly pressurized liquid carbon dioxide to above its critical point, such that supercritical carbon dioxide is formed;
sealing an extractable material with the supercritical carbon dioxide under pressurized conditions to produce a target compound laden supercritical carbon dioxide through high intensity extraction targeting;
releasing the pressurized target compound laden supercritical carbon dioxide from the extraction vessel in a controlled manner by flow regulation; and
collecting the target compound and producing carbon dioxide gas by coalescing the target compound from the target compound laden supercritical carbon dioxide through expansion cooling,
such that high intensity supercritical fluid extraction of a target compound is achieved.

19. The method of HIT supercritical fluid extraction of claim 18, further comprising the step of recycling the carbon dioxide gas by reducing the temperature of the carbon dioxide gas after collection of the target compound to recycle the carbon dioxide gas by producing recycled liquid carbon dioxide.

20. The method of HIT supercritical fluid extraction of claim 18, wherein the method is automated.
